(19) United States
(12) Patent Application Publication
(10) Pub. No.: US 2025/0018073 A1
(43) Pub. Date: Jan. 16, 2025

(54) CURTAIN AND WALL DISINFECTANT DEVICE

(71) Applicant: Robert DiNunzio, Cherry Hill, NJ (US)

(72) Inventor: Robert DiNunzio, Cherry Hill, NJ (US)

(21) Appl. No.: 18/766,817

(22) Filed: Jul. 9, 2024

Related U.S. Application Data
(60) Provisional application No. 63/526,494, filed on Jul. 13, 2023.

(51) Int. Cl. A61L 2/10; A61L 2/26

(52) U.S. Cl. CPC A61L 2/10; A61L 2/26; A61L 2202/11; A61L 2202/16; A61L 2202/25

(57) ABSTRACT

A portable hospital curtain and wall disinfectant device utilizes ultraviolet lamp lighting located within lamp masts affixed to telescopic support members. The telescopic support members allow the lamp masts to be vertically and horizontally adjustable in order to decontaminate and disinfect curtains and specific wall areas.

### RELATED APPLICATION

This application claims the benefit of application Ser. No. 63/526,494, filed on Jul. 13, 2023.

### FIELD OF THE INVENTION

The present invention relates to hygiene and safety, and specifically to the sanitization and decontamination of curtains and walls of hospitals and other facilities which must be kept germ free.

### BACKGROUND OF THE INVENTION

The cleanliness and sanitation of healthcare equipment, accessories, and patient spaces are imperative in hospital and similar medical environments. These conditions are especially critical in patient rooms and in hospital spaces where medical personnel treat patients.

In such areas, curtains are utilized for a variety of purposes, for instance, in operating and recovery rooms, in order to ensure privacy when multiple patients occupy a common space, etc. However, in a hospital or like healthcare facility where a clean and germ free environment is of primary importance, repeated contact of curtains by individuals presents an invitation for infection. Curtains will become contaminated as various users, including medical staff and visitors, grasp onto the edges of the curtains to open and close then during day-to-day operations. Since hospital curtains are expensive, they are rarely replaced and lack of time and resources prevent them from being laundered or changed between patients and usage. As a practical matter, in many cases, curtains are often never properly clean. Thus, these curtains become a prime source for the potential spreading of infection in environments that cannot afford this threat. Likewise, the spaces in which patients are being treated and are recovering are also subject to contamination. These areas, especially room walls, are particularly vulnerable to the formation of bacteria and other harmful germs.

### SUMMARY OF THE INVENTION

It is thus the object of the present invention to provide a disinfectant device for medical facilities to counter the formation of injurious bacteria and like germs on medical facility curtains and walls which become contaminated by these pathogens.

These and other objects are accomplished by the present invention, a portable hospital curtain and wall disinfectant device which utilizes ultraviolet lamp lighting located within lamp masts affixed to telescopic support members. The telescopic support members allow the lamp masts to be vertically and horizontally adjustable in order to decontaminate and disinfect curtains and specific wall areas.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 is a perspective view of the disinfectant device of the present invention with both mast lamps lowered onto the base members.

FIG. 2 is a perspective view of the disinfectant device of the present invention with both mast lamps in the fully raised, curtain cleaning position.

FIG. 3 is a perspective view of the disinfectant device of the present invention with the lamp masts positioned vertically for wall cleaning.

FIG. 4 is a perspective view of the disinfectant device of the present invention with the lamp masts positioned horizontally for wall cleaning.

### DETAILED DESCRIPTION OF THE INVENTION

The curtain and wall disinfectant device 1 of the present invention comprises horizontal base member 2 having wheels 4 which allow the device to be readily portable.

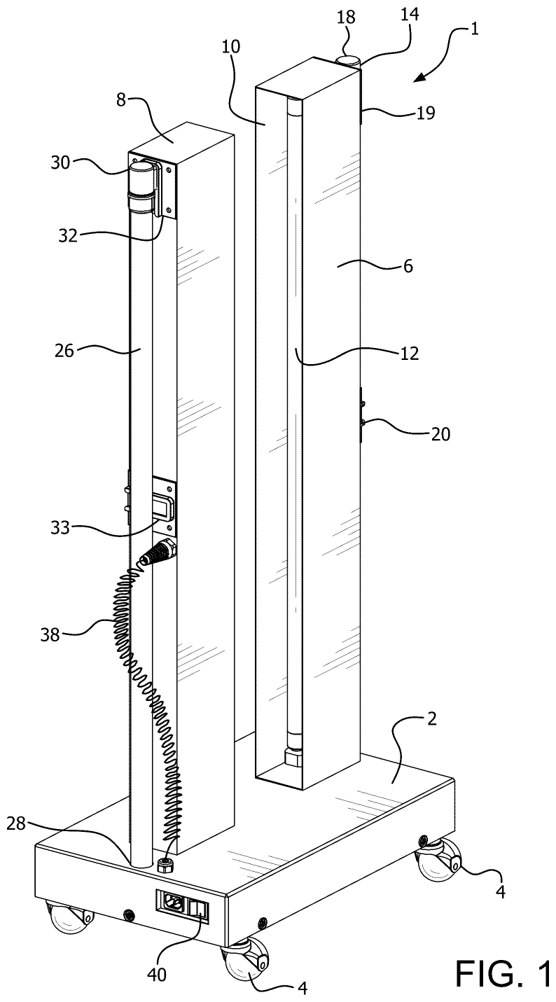

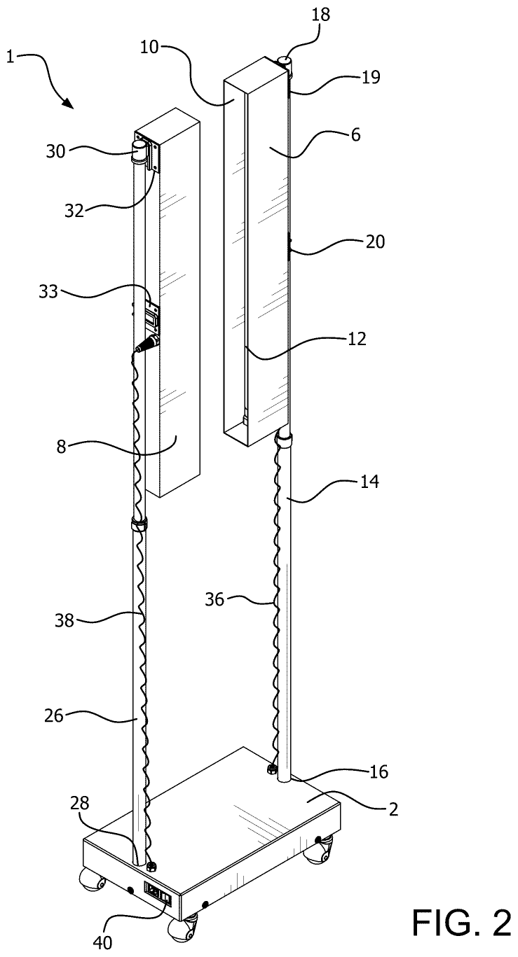

As seen in FIGS. 1 and 2, first lamp mast 6 extends vertically upward from base member 2. Lamp mast 6 comprises vertical space 10 within the lamp mast. Ultraviolet lamp 12 is located within space 10. Telescopic support member 14 has its lower end 16 connected to base member 2 and its upper end 18 inserted into bracket 19 connected to the top end of lamp mast 6. Upper end 18 of telescopic support member 14 fits snuggly into bracket 19. Bracket 20 is located at the approximate mid-section of lamp mast 6.

Also as seen in FIGS. 1 and 2, second lamp mast 8 extends vertically upward from base 2 and is identical to lamp mast 6. First lamp mast 8 and second lamp mast 20 are located on opposite sides of base member 2. Lamp mast 8 comprises vertical space 22 within the lamp mast. Ultraviolet lamp 24 is located within space 22. Telescopic support member 26 has its lower end 28 connected to base member 2 and its upper end 30 inserted into bracket 32 connected to lamp mast 8. Upper end 30 fits snugly into bracket 32. Telescopic support members 14 and 26 are located on opposite sides of base member 2. Bracket 33 is located at the approximate mid-section of telescopic support member 26.

Electricity is supplied to ultraviolet lamps 12 and 24 by electrical wiring 36 and 38, which extends from both lamp masts to base 2. Switch 40 serves to turn the electrical power on to illuminate lamps 12 and 24 and off to turn off the lamps.

Telescopic support members 14 and 26 are extendable to raise first lamp mast 6 and second lamp mast 8 vertically in relation to base member 2.

FIG. 1 illustrates the manner in which lamp masts 6 and 8 of disinfectant device 1 are positioned to disinfect and decontaminate the bottom section of a curtain. In use, the lower section of a curtain is simply positioned between mast lamp 6 and mast lamp 8. Ultraviolet lamps 12 and 24 are then illuminated, thereby allowing the ultraviolet lighting to affect the curtain. Once this area of the curtain is disinfected, wheels 4 on base 2 allow disinfectant device 1 to be moved laterally to treat an adjacent section of the curtain or telescopic support members can be raised in order to raise mast lamps 6 and 8, as seen in FIG. 2, to treat an upper section of the curtain. This process is continued until the entire curtain is disinfected and decontaminated.

FIG. 1 also illustrates the manner in which disinfectant device 1 will be set for storage when not in use.

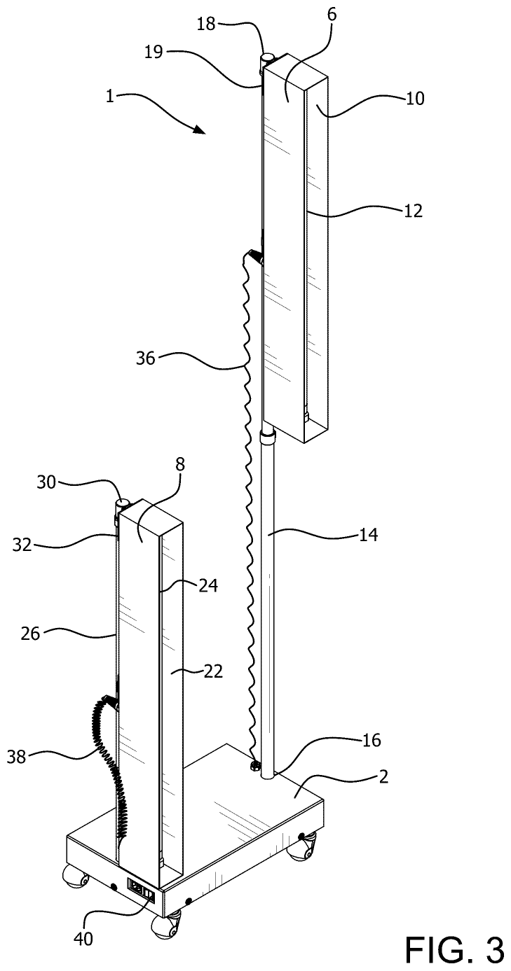

FIG. 3 illustrates the manner in which lamp masts 6 and 8 can be vertically positioned to disinfect and decontaminate wall areas. The lamp masts are easily rotated 90° simply by manually turning them within brackets 19 and 32.

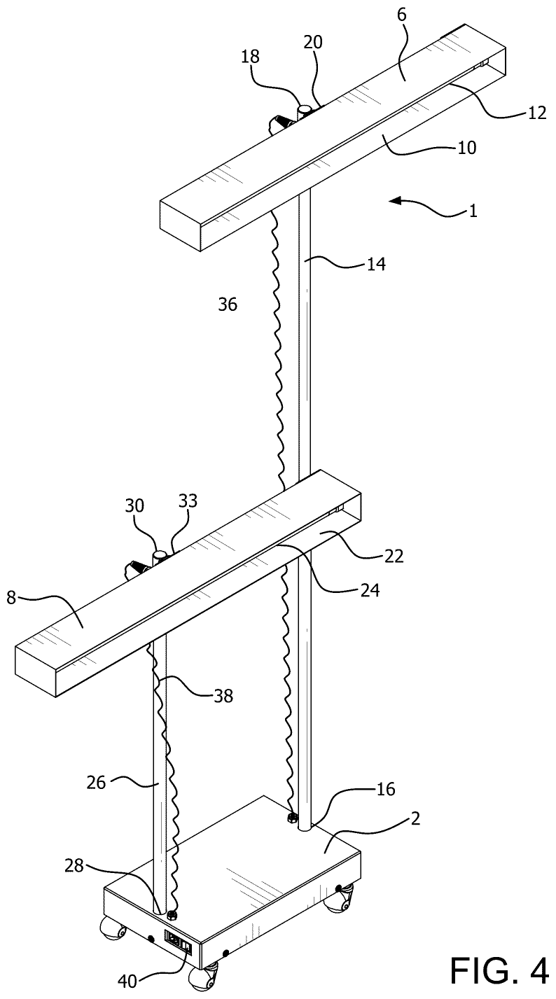

FIG. 4 illustrates the matter in which lamp masts 6 and 8 are horizontally positioned to disinfect and decontaminate wall areas. For this use, the lamp masts are removed from brackets 19 and 32 and telescopic support members 14 and 26 are inserted snugly into brackets 20 and 33, respectively. Once again after an area is disinfected, wheels 4 allow disinfectant device 1 to be moved to different areas of the wall for continued decontamination.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A curtain and wall disinfectant device comprising:
a base member;
a first lamp mast, said lamp mast comprising an upper end, a vertical space within the lamp mast, and an ultraviolet lamp located within the space;
a first telescopic support member having a lower end connected to the base member and an upper end;
a second lamp mast, said second lamp mast comprising an upper end, a vertical space within the lamp mast and an ultraviolet lamp located within the space; and
a second telescopic support member having a lower end connected to the base member and an upper end;
wherein the first and second telescopic support members are configured to be extendable to raise the first and second lamp masts in relation to the base member.

2. The curtain and wall disinfectant device as in claim 1 wherein the first and second telescopic support members are located on opposite sides of the base member.

3. The curtain and wall disinfectant device as in claim 1 wherein the base member is transportable by wheels attached to the base member.

4. The curtain and wall disinfectant device as in claim 1 wherein electricity is supplied to the ultraviolet lamps by electrical wiring which extends from both lamp masts to the base member.

5. The curtain and wall disinfectant device as in claim 1 further comprising a first bracket attached at the upper end of the first lamp mast and a second bracket attached at the mid-section of said first lamp mast, wherein the upper end of the telescopic support member is configured to be affixed to the first bracket to maintain the first lamp mast in a vertical position and the upper end of said telescopic support member is configured to be affixed to the second bracket to maintain the first lamp mast in a horizontal position.

6. The curtain and wall disinfectant device as in claim 1 further comprising a first bracket attached at the upper end of the second lamp mast and a second bracket attached at the mid-section of said second lamp mast, wherein the upper end of the telescopic support member is configured to be affixed to the first bracket to maintain the second lamp mast in a vertical position and the upper end of said telescopic support member is configured to be affixed to the second bracket to maintain the second lamp mast in a horizontal position.

7. The curtain and wall disinfectant device as in claim 6 further comprising a first bracket attached at the upper end of the first lamp mast and a second bracket attached at the mid-section of said first lamp mast, wherein the upper end of the telescopic support member is configured to be affixed to the first bracket to maintain the first lamp mast in a vertical position and the upper end of said telescopic support member is configured to be affixed to the second bracket to maintain the first lamp mast in a horizontal position.

8. The curtain and wall disinfectant device as in claim 1 further comprising a switch located on the base member for turning electrical power to the device on and off.
